(19) United States
(12) Patent Application Publication
(10) Pub. No.: US 2025/0032962 A1
(43) Pub. Date: Jan. 30, 2025

(54) DISPOSABLE FILTRATION DEVICE FOR PURIFYING ADIPOSE TISSUE

(71) Applicant: NEOSYAD, AIX-EN-PROVENCE (FR)

(72) Inventors: Régis ROCHE, LORGUES (FR); François CABAUD, EGUILLES (FR); Xavier NELISSEN, VILLERS SAINT SIMEON (BE)

(21) Appl. No.: 18/717,120

(22) PCT Filed: Nov. 4, 2022
(86) PCT No.: PCT/FR2022/052078

§ 371 Date: Jun. 6, 2024

(30) Foreign Application Priority Data
Dec. 8, 2021 (FR) FR2113140

(51) Int. Cl. B01D 33/11; B01D 33/60; B01D 33/72; B01D 33/80; B01D 36/00

(52) U.S. Cl. CPC B01D 33/11; B01D 33/60; B01D 33/72; B01D 33/801; B01D 33/805; B01D 36/00; B01D 2201/16

(57) ABSTRACT

The present invention relates to a disposable filtration device for purifying adipose tissue, which device comprises a sealed enclosure, a filter present in the sealed enclosure, said filter delimiting a centrifugation chamber for adipose tissue, pipes connected to the sealed enclosure,

and a rotary plate linked to the filter, the rotary plate being configured to cooperate with a rotary drive device.

### CROSS REFERENCE TO RELATED APPLICATIONS

This application claims priority to PCT Application no. PCT/FR2022/052078 filed on Nov. 4, 2022, which claims priority to FR2113140 filed on Dec. 8, 2021. The contents of each of which are incorporated by reference herein.

### TECHNICAL FIELD

The present application relates to a disposable filtration device for purifying adipose tissue. The application has application, in particular, in “lipofilling” for aesthetic or reconstructive purposes. The application can be used, in particular, in lipofilling useful for mammary surgery operations, the use of the device according to the application not however being limited to this application.

### PRIOR ART

Lipofilling operations or autologous grafting of adipose tissue are used in surgery, especially in surgery on the buttocks to increase volume or in mammary surgery in order to model the breast in order to give it a more natural appearance after a Deep Inferior Epigastric Perforator (DIEP), a reconstruction using a latissimus dorsi flap, or after the insertion of a mammary implant. These operations consist of taking fat from a donor region of a patient, in order to then reintroduce it into the region of interest in the patient's body.

Currently, these operations are not reproducible and may require several interventions due to the resorption of the introduced material (volume loss encountered after the operation). More specifically, the adipose tissue taken contains a liquid medium mainly consisting, on the one hand, of oil and blood initially present in the adipose tissue and, on the other hand, a liquid such as a physiological solution introduced during the taking of the adipose tissue and/or during a washing operation thereof.

The inventors have determined that the rate of resorption is mostly linked to the quantity of liquid present in the adipose tissue reintroduced into the body of the patient. In other words, the more liquid which remains in the reinjected adipose tissue, the higher the rate of resorption. It is therefore important to treat the adipose tissue before its reintroduction into the body of the patient, in order to remove as much of the liquid medium as possible. However, this treatment must be carried out under hygienic conditions in order to eliminate the risk of contamination of one patient by another.

Document US 2020/0054824 discloses a lipofilling system which uses a gravity filtration device marketed under the tradename Revolve®. However, this type of filtration device does not allow a sufficient quantity of liquid to be removed from the adipose tissue so as to be able to significantly reduce the rate of resorption. Furthermore, in the system of document US 2020/0054824, the adipose tissue circulates in a pumping circuit which must be disinfected between two operations, which leads to the system being immobilised after each transplant. In addition it is difficult to control the disinfection in the entire pumping circuit.

However, there is a need for a solution which enables an optimum treatment for removing the liquid medium present in adipose tissue to be performed, while ensuring very good hygiene conditions.

### DISCLOSURE

For this purpose, the present application proposes a disposable filtration device for purifying adipose tissue, comprising at least:
a sealed enclosure;
a filter present in the sealed enclosure, said filter delimiting a centrifugation chamber for adipose tissue;
pipes connected to the sealed enclosure;
a rotary plate linked to the filter, the rotary plate being configured to cooperate with a rotary drive device.

The filtration device of the application thus advantageously combines the use of a filter that is able to drain a liquid medium from the adipose tissue with setting it in rotation. More specifically, since the filter forms a centrifugation chamber, adipose tissue present inside this chamber can be subjected to centrifugal accelerations against the inner wall of the filter. The liquid medium present in and around the adipose tissue is then effectively drained to the outside of the centrifugation chamber, through the pores of the filter, while the adipose tissue is retained in the chamber. Thus, a large quantity of liquid is removed, allowing purified adipose tissue to be obtained which, once re-implanted, has a resorption rate much lower than that obtained with filtration solutions of the prior art.

Furthermore, the filtration device of the application comprises pipes which are used for the circulation of the adipose tissue. The pipes being part of the disposable device, perfect hygiene is ensured for each intervention through the replacement of the elements in which the adipose tissue has circulated or passed.

In an embodiment, the purification device further comprises a stiffening element present between the sealed enclosure and the filter.

In another exemplary embodiment, the filter is made of a rigid self-supporting material. In this case, a stiffening element is not necessary in the purification device.

In an embodiment, the purification device further comprises a collector plate present in the centrifugation chamber, the collector plate being able to move in translation along the axis of rotation of the filter. The collector plate acts as a piston which can scrape the inner wall of the filter so as to collect a maximum of adipose tissue and to facilitate the collection of the purified adipose tissue at the top of the device. According to another particular aspect, the purification device further comprises a threaded rod extending in the centrifugation chamber and cooperating with a tapped portion of the collector plate, the threaded rod being connected to rotary drive means of the filter, said means being configured to drive the threaded rod in rotation, in a direction of rotation opposite to the direction of rotation of the filter. Here, the same rotary drive means is advantageously used both for the centrifugation of the filter and for the movement in vertical translation of the collector plate.

According to another particular aspect, the purification device further comprises a protective sleeve surrounding the threaded rod. This sleeve can prevent the threaded rod from coming into contact with the adipose tissue which by accumulating at the points where the plate is tapped, can block the movement thereof.

Another object of the application is a machine for purifying adipose tissue from a patient, the machine comprising at least:
a disposable filtration device according to the application,
a rotary drive device on which the disposable filtration device is removably fixed, the rotary drive device comprising a rotary motor connected to the disposable filtration device,
a suction pump connected to the disposable filtration device,
a circulation pump connected to the disposable filtration device,
a reservoir of washing liquid connected to the disposable filtration device,
a control unit configured to control the rotary drive device, the suction pump and the circulation pump.

The machine of the application can perform all the necessary operations (collection, washing, centrifugation and reimplantation of adipose tissue) in an adipose tissue transplant treatment, and this under very good hygiene conditions for each treatment, through the use of a disposable filtration device.

In an exemplary embodiment, the machine further comprises a pinch valve device cooperating with pipes of the disposable filtration device, the control unit being configured to control said pinch valve device so as to selectively prevent the circulation in one or more pipes.

The pinch valve device can selectively control the circulation in the pipes, while facilitating the assembly and disassembly of the pipes during the replacement of the disposable filtration device.

In an embodiment, the rotary drive device comprises a stator flange, the sealed enclosure of the disposable filtration device comprising removable fixation means able to lock the rotation of said sealed enclosure on the stator flange.

It is thus possible quickly and easily to lock and unlock a disposable filtration device on the machine.

### BRIEF DESCRIPTION OF THE FIGURES

FIG. 1 is a schematic perspective view of a machine for purifying adipose tissue, in accordance with an embodiment of the application.

FIG. 2 is an exploded view of a disposable filtration device in accordance with an exemplary body of the application,

FIG. 3 is a schematic sectional view of the disposable filtration device of FIG. 1, once assembled,

FIG. 4 is a schematic sectional view showing the disposable filtration device of FIG. 2 after movement of the collector plate,

FIG. 5 is a schematic sectional view showing the disposable filtration device of FIG. 2 before fixation on a rotary drive device of the machine for purifying adipose tissue,

FIG. 6 is a schematic sectional view showing the disposable filtration device of FIG. 2 after fixation on a rotary drive device of the machine for purifying adipose tissue,

FIG. 7 is a detailed view of a pinch valve device of the machine for purifying adipose tissue, of FIG. 1.

### DESCRIPTION OF THE EMBODIMENTS

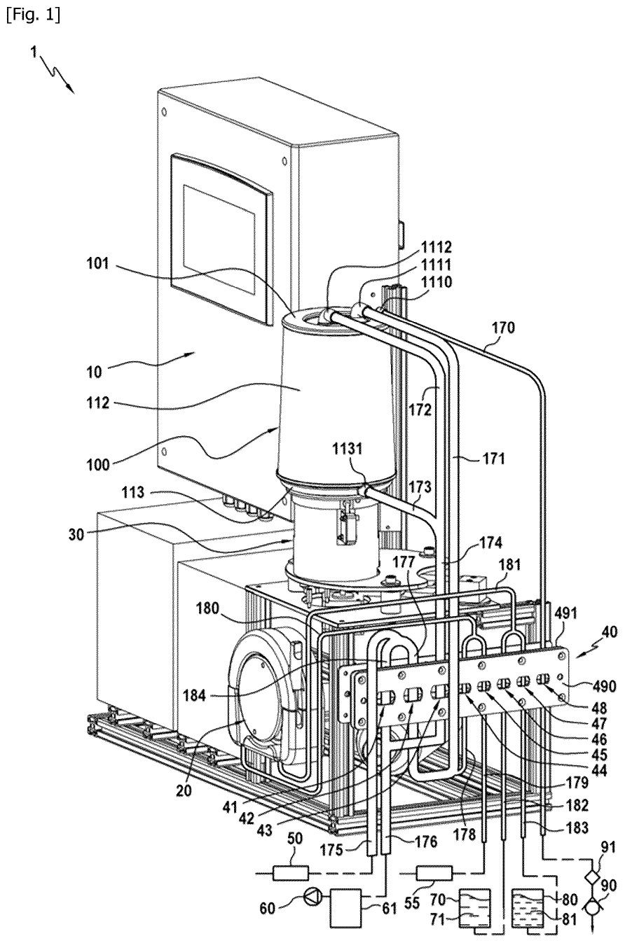

FIG. 1 illustrates an exemplary machine 1 according to the application for purifying adipose tissue. In a general manner and as will be described in more detail below, the machine 1 comprises a control unit 10 enabling the implementation of the various treatment steps, a circulation pump 20 and a suction pump 60 controlled by the control unit 10, and a rotary drive device 30 controlled by the control unit 10. The machine 1 further comprises a disposable filtration device 100 for purifying adipose tissue in accordance with an embodiment of the application, the filtration device 100 being removably mounted on the machine 1.

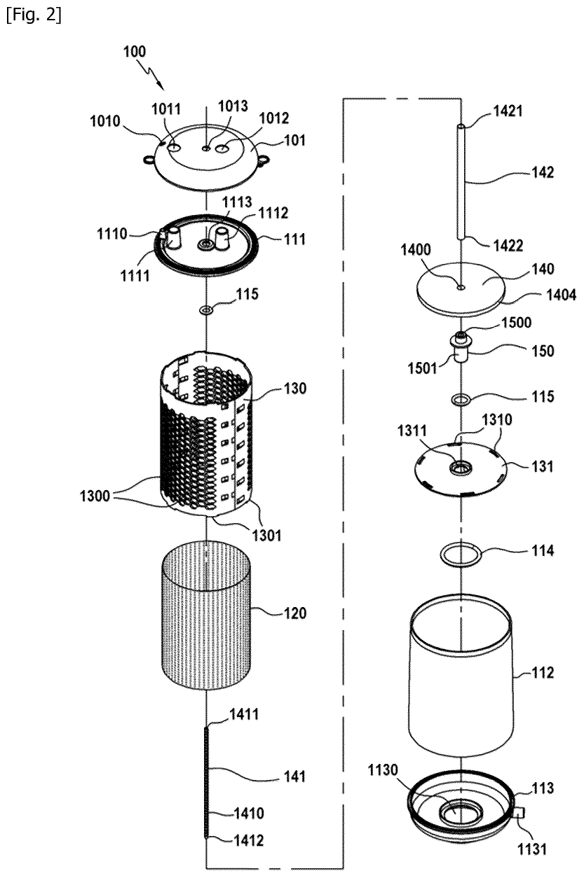

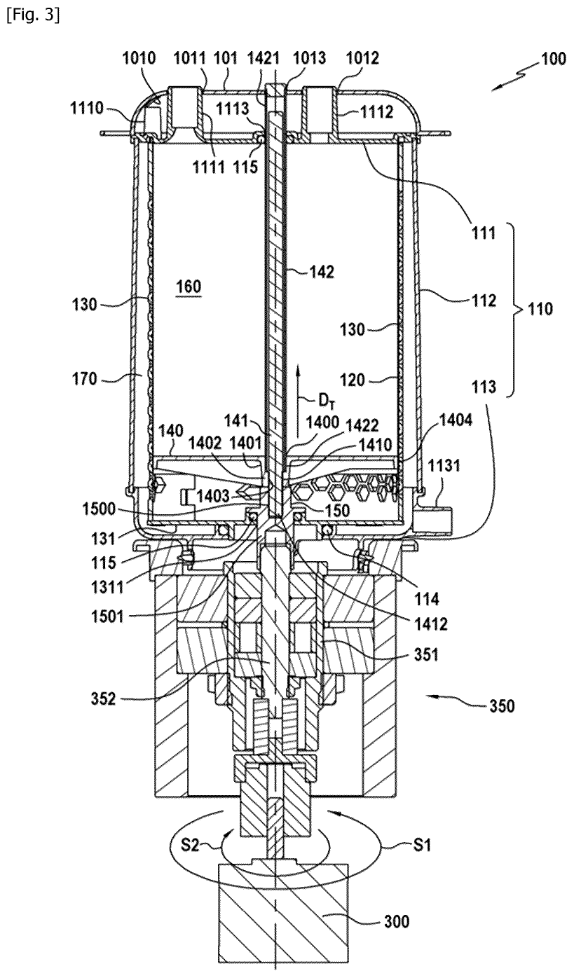

As illustrated in FIGS. 2 and 3, the disposable filtration device 100 comprises, in particular:
a sealed enclosure 110,
a filter 120 present in the sealed enclosure 110, said filter delimiting a centrifugation chamber 160 for adipose tissue,
a plurality of pipes 170 to 173, connected to the sealed enclosure 110,
a rotary plate 131 linked to the filter 120, the rotary plate being configured to cooperate with the rotary drive device 30.

The sealed enclosure 110 is formed here by a cap 111, a cylindrical wall 112 and a bottom 113. These elements are fixed together in a sealed manner.

The filtration device 100 also comprises a filter or screen 120 present in the sealed enclosure 110. The filter 120 delimits a centrifugation chamber 160 (FIG. 3), the operation of which is described below. In the example described here, the filter 120 has a cylindrical shape. The filter can have other shapes suitable for centrifugation. The filter 120 has a pore size that is configured to allow a liquid medium to pass and to retain adipose tissue. The pore size is chosen, in particular, to enable the passage of liquids such as oil, blood, water or a physiological solution, while retaining the adipose tissue. By way of non-limiting examples, the pore size of the filter can be between 50 μm and 1500 μm, and more preferably between 200 μm and 500 μm. The filter can be produced in different ways, for example by braiding, welding plastic threads or from a material comprising apertures or holes having dimensions corresponding to the desired size. The filter can, in particular, be made of a polymer material such as polyester or polypropylene but a person skilled in the art will know that other materials can be used.

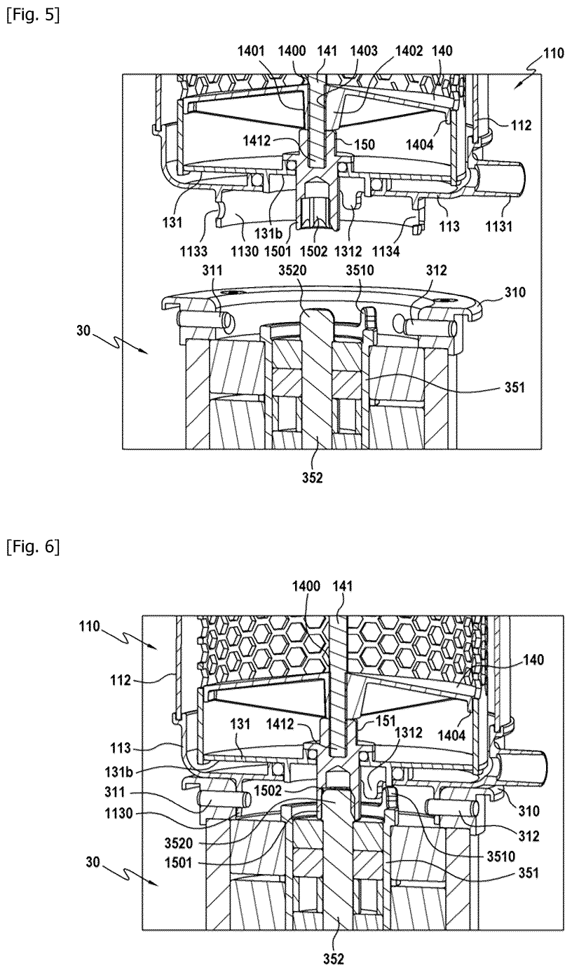

In the example described here, a cylindrical stiffening element 130 is present between the cylindrical wall 112 of the sealed enclosure 110 and the filter 120. Its function, in particular, is to ensure the structural stability of the filter 120 during centrifugation. The stiffening element 130 is made, for example, from metal or plastic material and has a perforated structure defining a plurality of openings 1300 in order to enable the evacuation of the liquid medium drained by the filter 120. The stiffening element 130 is associated with a rotary plate 131. More precisely, at its lower end, the stiffening element 130 has teeth 1301 which cooperate with grooves 1310 that are present in the vicinity of the outer periphery of the rotary plate 131. Of course, any means of securing the stiffening element with the rotary plate, such as clipping or gluing, for example is possible. The rotary plate 131 is connected to the rotary drive device 30. The rotary drive device comprises a rotary electric motor 300. The electric motor 300 can be, for example, a stepper motor or a brushless DC motor. In the example described here, the rotary plate 131 is connected to the rotary electric motor 300 by a bidirectional clutch 350 configured to ensure, via a first shaft 351, the driven rotation of the rotary plate 131 in a first direction of rotation S₁ of the motor 30 (FIG. 3). The bidirectional clutch can be replaced by any device enabling a selective driven rotation in two opposite directions of rotation. Seals 114 and 115 are respectively placed below and above the rotary plate 131 in order to ensure the sealing in the lower part of the purification device. The filtration device 100 being disposable, it is mounted interchangeably on the machine. For this purpose and as illustrated in FIGS. 3 to 6, the rotary drive device 30 comprises a stator flange 310 on which the bottom 113 of the sealed enclosure 110 is fixed. In the example described here, a bayonet type fixation is used, which enables both securing of the disposable filtration device 100 with the stator flange 310 of the rotary drive device 30 of the machine 1, and locking of the rotation of the sealed enclosure 110 of the device. More precisely, the bottom 113 of the sealed enclosure 110 comprises a fixation ring 1130 which has two notches 1133 and 1134 which cooperate with two tenons 311 and 312 present on the stator flange 310. It is thus possible quickly and easily to lock and unlock a disposable filtration device 100 on the machine 1. Any other fixation system can be used to secure the disposable filtration device on the rotary drive device 30 and to lock the rotation of the sealed enclosure 110 of the disposable filtration device 100.

In the example described here, the rotary plate 131 comprises a finger 1312 which extends from the inner face 131b of the rotary plate and which is intended to interact with another finger 3510 present on the end of the first shaft 351 connected to the rotary motor 30 when the filtration device 100 is mounted on the machine (FIG. 6). Any other system enabling a coupling of the rotary drive device 30 with the rotary plate 31 can be used.

The centrifugation is performed by rotating the filter 120. More precisely, the electric motor 300 is controlled by the control unit 10 in a first direction of rotation S₁ in order to drive the rotary plate 131 and the stiffening element 130 into engagement with the plate 131. The rotation of the plate 131 and of the stiffening element 130 drives the rotation of the filter 120. The filter is fixed to the stiffening element by any type of suitable means. By way of non-limiting examples, the filter can be fixed using one or more of the following means: glueing, engagement in wedging notches present on the stiffening element, clipping. The speed of the electric motor 30 is controlled by the control unit 10 so as to apply a centrifugal acceleration to the adipose tissue present in the centrifugation chamber. Thus, adipose tissue present in the centrifugation chamber 160 will be subjected to a centrifugal force against the inner wall of the filter 120, which makes it possible to effectively drain the liquid medium present in the adipose tissue without damaging this.

The disposable filtration device comprises at least four pipes 170, 171, 172 and 173, pipes 170, 171 and 172 being respectively connected to ports 1110, 1111, 1112 present on the cap 111 of the sealed enclosure 110, while pipe 173 is connected to an evacuation port 1131 present on the bottom 113 of the enclosure 110. The other end of pipe 170 is connected to a venting device 90 equipped with a filter 91. In the example described here, as illustrated in FIG. 1, other pipes are used in order to form circulation circuits in the machine 1 enabling the performance of various steps of the treatment for purifying adipose tissue.

More precisely, in the embodiment described here, the machine 1 further comprises:
a pipe 174 joining pipes 172 and 173 connected to the filtration device 100,
a pipe 175, one end of which is connected to a suction cannula 50 intended to suck adipose tissue on a patient,
a pipe 176, one end of which is connected to a suction pump 60, pipe 176 opening into pipe 174,
a pipe 177 connecting pipe 175 and a pipe 184 to pipe 171,
a pipe 178 connecting pipes 171 and 177 to pipes 180 and 179,
pipe 179, one end of which is connected to an injection cannula 55 intended to deliver an anaesthetic and purified adipose tissue into the body of the patient, pipe 179 being connected to pipes 178 and 180,
pipe 180, one end of which is connected to the circulation pump 20, pipe 180 being connected to pipes 178 and 179,
a pipe 181, one end of which is connected to the circulation pump 20, pipe 181 being connected to pipes 182 and 183,
pipe 182, one end of which is connected to a reservoir of washing liquid 70, pipe 182 being connected to pipe 181 and to a pipe 183,
pipe 183, one end of which is connected to a reservoir of washing liquid and anaesthetic 80, pipe 183 being connected to pipes 181 and 182,
a pipe 184 connecting pipes 175 and 177 with pipe 174.

All the pipes described above are disposable.

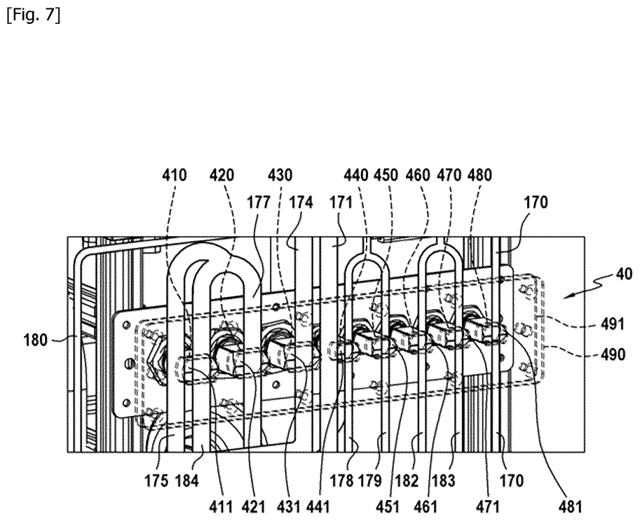

The machine 1 further comprises a pinch or throttling valve device 40 comprising a plurality of pinch modules 41 to 48, also called a pinch valve, capable of selectively pinching one or more pipes described above, in order to prevent circulation, and capable of releasing the pinching in order to allow this circulation. In the example described here and as illustrated in FIG. 7, the pinch modules 41 to 48 each comprise a housing 410 to 480 delimited between two plates 490 and 491 and in which a pipe is disposed. The pinch modules 41 to 48 each further comprise a finger 411 to 481 that can move in the corresponding housing, between a retracted position (open position of the module), in which circulation is authorised in the pipe present in the housing, and a deployed position (closed position of the module), in which the finger pinches the pipe present in the housing, thus preventing circulation in the pipe.

In the example described here:
pipe 184 is present in housing 410 of pinch module 41,
pipe 177 is present in housing 420 of pinch module 42,
pipe 174 is present in housing 430 of pinch module 43,
pipe 178 is present in housing 440 of pinch module 44,
pipe 179 is present in housing 450 of pinch module 45,
pipe 182 is present in housing 460 of pinch module 46,
pipe 183 is present in housing 470 of pinch module 47,
pipe 170 is present in housing 480 of pinch module 48.

The position of each pinch module will now be described as a function of the steps or sequences implemented by the machine 1.

In the anaesthesia step, the control unit 10 places pinch modules 45 and 47 in the open position authorising circulation in pipes 179 and 183, and places pinch modules 41, 42, 43, 44, 46 and 48 in the closed position, preventing circulation in pipes 184, 177, 174, 178, 182 and 170. Through action on the pump 20 by the control unit 10, a liquid 81 present in the reservoir of washing liquid and anaesthetic 80 is sucked up by the pipe 183. The liquid is conveyed into the pump 20 via pipe 181, then is discharged therefrom by pipe 180 and pipe 179 in order to reach the injection cannula 55 used for introducing the anaesthetic into the body of the patient.

In the step of collecting adipose tissue from the body of a patient, the control unit 10 places pinch modules 42 and 43 in the open position authorising circulation in pipes 177 and 174, and places pinch modules 41, 44, 45, 46, 47 and 48 in the closed position, preventing circulation in pipes 184, 178, 179, 182, 183 and 170. The operation of the suction pump 60 by the control unit 10 enables a vacuum to be drawn in the filtration device 100 via pipes 176, 174, 173 and 172, which provokes suction of the adipose tissue by the suction cannula 50 and its conveying into the filtration device 100 via pipes 175, 177 and 171.

In the step of purifying the collected adipose tissue, the control unit 10 places pinch modules 44 and 46 in the open position authorising circulation in pipes 178 and 179, and places pinch modules 41, 42, 43, 45, 47 and 48 in the closed position, preventing circulation in pipes 184, 177, 174, 179, 183 and 170. Through the operation of the pump 20 by the control unit 10, a liquid 71 present in the reservoir of washing liquid 70 is sucked up by pipe 182. The liquid is conveyed into the pump 20 via pipe 181, then is discharged therefrom by pipe 180, pipe 178 and pipe 171, in order to reach the filtration device 100. During the introduction of the washing liquid into the filtration device 100, one or more centrifugation cycles are performed, as described above, in order to separate the contaminant materials. The evacuation of the washing liquid containing the contaminant materials (water, blood, oil, etc.) of the adipose tissue is performed by actuating the suction pump 60 and placing pinch modules 41, 43 and 48 in the open position by the control unit 10. In this configuration, the washing liquid containing the contaminant materials is sucked up by the evacuation port 1131 of the filtration device 100, then circulates in pipes 173, 174 and 176, in order to be conveyed into a waste bin 61 present upstream of the suction pump 60. The placing of module 48 in the open position, permits the venting of the filtration device and thus facilitates the evacuation of the washing liquid containing the contaminant materials. The treatment for purifying adipose tissue with the disposable device of the application is then achieved.

The pinch valve device 40 can selectively control the circulation in the pipes, while facilitating the assembly and disassembly of the pipes during the replacement of the disposable filtration device.

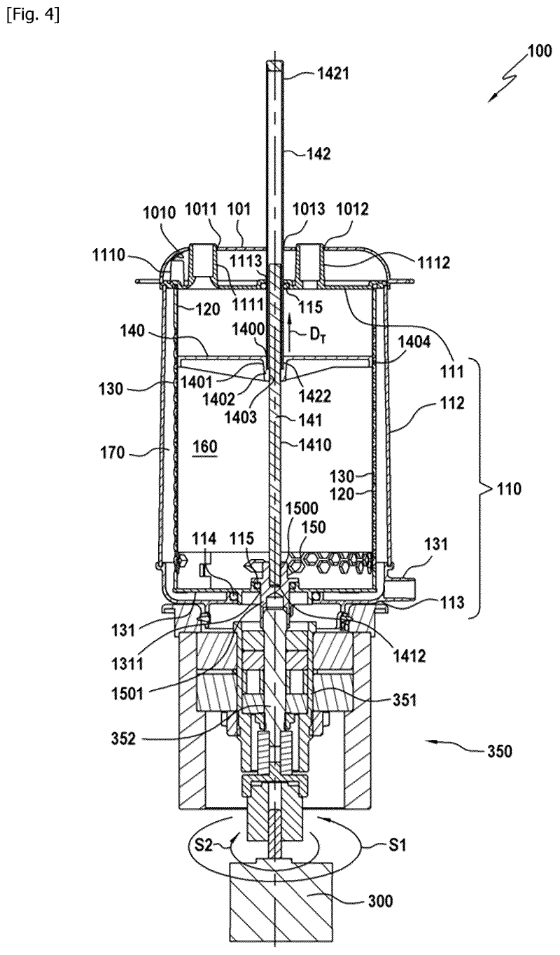

Any other device making it possible to selectively prevent the circulation of material in the pipes could be used to perform the various sequences described above. Optionally, the purification device can further comprise a collector plate that can move inside the centrifugation chamber. As illustrated in the examples of FIGS. 3 and 4, the purification device 100 further comprises a collector plate 140 which can move in translation in a direction Dr along the axis X of the filter 120. More precisely, the purification device 100 comprises a threaded rod 141 which extends vertically inside the centrifugation chamber 160 following the axis X of the filter 120. The lower end 1412 of the threaded rod is connected to the electric motor 10 by means of a guide 150 and the bidirectional clutch20. The guide 150 comprises a first housing 1500 in which the lower end 1412 is fixed. A lower portion 1501 of the guide 150 comprises a second housing 1502 which cooperates with the free end 3520 of a second shaft 352 of the bidirectional clutch 350 when the disposable filtration device 100 is mounted on the rotary drive device 30 (FIG. 6). The second shaft 352 is engaged with the electric motor 300 when this transmits a rotary movement in a second direction of rotation S₂ opposite to the first direction of rotation S₁ used for the centrifugation. When the electric motor 300 turns in the first direction of rotation S₁, no rotary movement is transmitted by the bidirectional clutch 350 to the guide 150 to which the threaded rod 141 is connected. Similarly, when the motor 300 turns in the second direction of rotation S₂, no rotary movement is transmitted by the bidirectional clutch 350 to the rotary plate 131 and, consequently, to the filter 120. On its upper face, the collector plate 140 has a central opening 1400 prolonged by a neck 1401 which extends from the inner face of the plate 140. The neck 1401 comprises a portion 1402 comprising a tapping 1403 which cooperates with a thread 1411 of the threaded rod 141. Thus, when the threaded rod is rotated in the second direction of rotation S₂, the collector plate 140 rises in the centrifugation chamber 160 in the direction D_{T}.

The peripheral edge 1404 of the collector plate 140 is opposite the inner wall of the filter 120. Thus, when the collector plate 140 is moved in vertical translation in direction D_{T}, it acts as a piston which can scrape the inner wall of the filter so as to collect as much adipose tissue as possible. The vertical translation of the plate 140 also makes it possible to place the adipose tissue collected as close as possible to the cap 111, and consequently to facilitate its collection for reimplantation.

According to a particular aspect, the threaded rod 141 can be housed in a protective sleeve 142. The protective sleeve 142 which extends between an upper end 1421 and a lower end 1422 can prevent the threaded rod 141 from coming into contact with the adipose tissue which by accumulating at the tapping 1403 in the collector plate 140 can block the movement thereof.

FIG. 4 shows the purification device 100 after operation of the electric motor 10 in the second direction of rotation S₂ enabling the driven rotation of the threaded rod 141. The rotation of the threaded rod 141 drives the vertical translation of the collector plate 140 in the direction D_{T}.

The lower end 1422 of the protective sleeve is fixed in the opening 1400 of the collector plate 140. In order to enable the movement of the protective sleeve 142 during movement of the plate 140, the cap 111 and the cover 101 respectively comprise an opening 1113 and an opening 1013, through which the sleeve 142 slides. A seal 115 is present around the opening 1113 in order to maintain the sealing at the top of the centrifugation chamber.

The collector plate can also be used in the step of reintroducing the purified adipose tissue into the body of the patient. In this case, the collector plate acts as a piston which pushes the purified adipose tissue towards the upper part of the centrifugation chamber as close as possible to the port 1111. The control unit 10 places the pinch modules 44 and 45 in the open position authorising the circulation of the adipose tissue in pipes 171, 178 and 179 and the pinch modules 41, 42, 43, 46, 47 and 48 in the closed position. The purified adipose tissue can thus be conveyed from the centrifugation chamber to the injection cannula 55.

In another exemplary embodiment, the filter is made of a rigid self-supporting material. The filter can be produced, in particular, from a metal strip in which holes are produced by laser, water jet or chemical cutting in order to form a screen, a metal grid woven with wires, or beads agglomerated by sintering of metal or ceramic powders. In this case, the stiffening element 130 is no longer necessary and the self-supporting filter is directly engaged with the rotary plate 131.

1. A disposable filtration device for purifying adipose tissue, comprising at least:
a sealed enclosure;
a filter present in the sealed enclosure, said filter delimiting a centrifugation chamber for adipose tissue;
pipes connected to the sealed enclosure; and
a rotary plate linked to the filter, the rotary plate being configured to cooperate with a rotary drive device.

2. The device according to claim 1, further comprising a stiffening element present between the sealed enclosure and the filter.

3. The device according to claim 1, wherein the filter is made of a rigid self-supporting material.

4. The device according to claim 1, 3, further comprising a collector plate present in the centrifugation chamber, the collector plate being able to move in translation along the axis of rotation of the filter.

5. The device according to claim 4, further comprising a threaded rod extending in the centrifugation chamber and cooperating with a tapped portion of the collector plate, the threaded rod being configured to cooperate with a rotary drive.

6. The device according to claim 5, further comprising a protective sleeve surrounding the threaded rod.

7. A machine for purifying adipose tissue, the machine comprising at least:
a disposable filtration device;
a rotary drive device on which the disposable filtration device is removably fixed, the rotary drive device comprising a rotary motor connected to the disposable filtration device;
a suction pump connected to the disposable filtration device;
a circulation pump connected to the disposable filtration device;
a reservoir of washing liquid connected to the disposable filtration device;
a control unit configured to control the rotary drive device, the suction pump and the circulation pump.

8. The machine according to claim 7, further comprising a pinch valve device cooperating with pipes of the disposable filtration device, the control unit being configured to control said pinch valve device so as to selectively prevent the circulation in one or more pipes.

9. The machine according to claim 7, wherein the rotary drive device comprises a stator flange, the sealed enclosure of the disposable filtration device comprising a removable fixation means able to lock the rotation of said sealed enclosure on the stator flange.

10. A disposable filtration device for purifying adipose tissue, comprising at least:
a sealed enclosure; and
a filter present in the sealed enclosure, said filter delimiting a centrifugation chamber for adipose tissue.

11. The disposable filtration device of claim 10, further comprising:
a rotary plate linked to the filter, the rotary plate being configured to cooperate with a rotary drive device.

12. The disposable filtration device of claim 10, further comprising:
one or more pipes connected to the sealed enclosure.

13. The disposable filtration device of claim 10, further comprising pipes connected to the sealed enclosure.
